(19) United States
(12) Patent Application Publication
(10) Pub. No.: US 2023/0384334 A1
(43) Pub. Date: Nov. 30, 2023

(54) METHOD FOR CALCULATING DRINKING TIME

(71) Applicant: Shanxi Medical University, Taiyuan (CN)

(72) Inventors: Keming Yun, Taiyuan (CN); Lele Wang, Taiyuan (CN); Wei Zhang, Taiyuan (CN); Ruilong Wang, Taiyuan (CN); Yongli Guang, Taiyuan (CN); Daming Zhang, Taiyuan (CN); Chao Zhang, Taiyuan (CN); Meng Hu, Taiyuan (CN); Zhiwen Wei, Taiyuan (CN); Wenfang Zhang, Taiyuan (CN); Zhongyuan Guo, Taiyuan (CN)

(21) Appl. No.: 18/449,654

(22) Filed: Aug. 14, 2023

Related U.S. Application Data
(63) Continuation of application No. PCT/CN2023/075150, filed on Feb. 9, 2023.

(30) Foreign Application Priority Data
May 16, 2022 (CN) 202210530167.3

(51) Int. Cl. G01N 33/98

(52) U.S. Cl. CPC G01N 33/98

(57) ABSTRACT

A method for calculating drinking time includes: drawing a plurality of blood samples within 0 to 120 h upon start of drinking, testing concentrations of alcohol, EtG and EtS in the blood samples, and obtaining an average concentration ratio C_{EtG}/C_{EtS}; obtaining a quadratic regression equation by fitting using the average concentration ratio C_{EtG}/C_{EtS} as an abscissa and sampling time as an ordinate; and measuring C_{EtG}/C_{EtS} of blood samples under test, obtaining a relationship between the drinking time and the C_{EtG}/C_{EtS} based on the quadratic regression equation, and calculating the drinking time.

### CROSS REFERENCE TO RELATED APPLICATIONS

This application is a continuation of PCT/CN2023/075150, filed on Feb. 9, 2023 and claims priority of Chinese Patent Application No. 202210530167.3, filed on May 16, 2022, the entire contents of which are incorporated herein by reference.

### TECHNICAL FIELD

The present disclosure relates to technical fields of analytical chemistry and judicial expertise, and in particular, relates to a method for calculating drinking time.

### BACKGROUND

Alcohol, as a psychoactive substance with dependency properties, has been widely abused worldwide. The number of alcohol-related accidents, such as violent behaviors, traffic accidents caused by alcohol abuse, has been increasing due to long-term excessive alcohol consumption by the general population. Accordingly, the technical identification of alcohol remains one of the most frequently encountered tasks in identification works of public security organs in China.

A metabolic process of the alcohol in the body is mainly accomplished through oxidative reactions (90-92%) of alcohol dehydrogenase (ADH) and acetaldehyde dehydrogenase (ALDH). In addition, a small amount of alcohol (<1%) perform non-oxidative metabolism under actions of different enzymes, and non-oxidative metabolites are produced directly combined with other substances. For example, Ethyl glucuronide (EtG) is a combination of the alcohol and glucuronic acid catalyzed by UDP-glucuronyltransferase. Moreover, the alcohol also can be combined with other sulphates to react under the action of sulfotransferase, thus producing Ethyl sulphate (EtS). As two major non-oxidative products for metabolizing the alcohol, EtG and EtS are present in small amounts but have long detection window periods, and they can be detected in a variety of body fluids or tissues even if the alcohol has been fully metabolized. Accordingly, EtG and EtS are expected to be sensitive and specific biomarkers for identifying alcohol intake.

Drinking time is an important clue in the analysis of alcohol-related cases and is of great significance in assessing the nature of the case. Therefore, inference of the drinking time is also one of the more common contents in the identification of alcohol-related cases.

Generally, effects of doses can be eliminated by calculating ratios. In recent years, studies on inference of dosing time by variation patterns in ratios of elementary bodies to metabolites or metabolites to metabolite concentrations over time have emerged constantly. However, it has been shown that the alcohol is rapidly metabolized in living bodies, and it is more difficult to detect the presence of alcohol after 8 h of drinking. Moreover, postmortem alcohol concentrations may change due to postmortem redistribution and postmortem generation. Accuracy for alcohol concentration detection is only recognized within 24 h after death and at temperatures below 20 DEG C. Therefore, the application of the concentration ratio of alcohol elementary bodies to metabolites has great limitations, and a more reliable method is needed for inferring the drinking time. EtG and EtS, as non-oxidative metabolites of alcohol, have higher concentrations and longer detection window periods, so that even if alcohol itself cannot be detected after alcohol intake, cases can be preliminarily judged by the detection of EtG and EtS. Moreover, relevant literature has proved that EtG and EtS are not produced postmortem and are relatively stable under low temperature conditions. Therefore, the concentration ratio of EtG to EtS can be considered to estimate the time of the last drinking.

### SUMMARY

In order to solve the above technical problems, the present disclosure is intended to provide a method for calculating drinking time. In the method according to the present disclosure, the drinking time is inferred using a variation pattern of concentration ratios between non-oxidative metabolites of alcohol over time, such that inevitable external interference in traditional methods is avoided.

Accordingly, some embodiments of the present disclosure provide a method for calculating drinking time.

The method includes:

drawing a plurality of blood samples within 0 to 120 h upon start of drinking, testing concentrations of EtG and EtS in the blood samples, and obtaining an average concentration ratio C_{EtG}/C_{EtS} of EtG to EtS;

obtaining a quadratic regression equation: y=1.646x²−0.9599x+0.0878, R²=0.9904 by fitting using the average concentration ratio C_{EtG}/C_{EtS} as an abscissa and sampling time as an ordinate, wherein x represents the average concentration ratio C_{EtG}/C_{EtS}, and y represents the sampling time; and

measuring C_{EtG}/C_{EtS} of blood samples under test, obtaining a relationship between the drinking time and the C_{EtG}/C_{EtS} based on the quadratic regression equation, and calculating the drinking time.

In some embodiments, a blood alcohol concentration upon drinking is in the range of 0.22 to 0.66 mg/m.

In some embodiments, an alcohol intake amount is 0.72 g/kg.

In some embodiments, sampling intervals of the blood samples used for obtaining the quadratic regression equation are at 0 h, 0.5 h, 2 h, 3 h, 5 h, 8 h, 12 h, 24 h, 36 h, 48 h, and 120 h respectively.

In some embodiments, the concentrations of EtG and EtS in the blood samples are tested by:

S1. pre-treating the blood samples

transferring the blood samples into centrifuge tubes added with internal standards EtG-D₅ and EtS-D₅, adding 80% of acetonitrile in methanol, precipitating and centrifuging at 0 DEG C., transferring supernatant, drying, re-dissolving with 5% of acetonitrile in water, centrifuging again, and taking the supernatant to obtain the blood samples under test; and

S2. measuring concentrations of EtG and EtS by liquid chromatography-tandem mass spectrometry for the blood samples under test in S1.

In some embodiments, in S2, a separation condition for liquid chromatography includes the following parameters:

chromatographic column: an Inertsil ODS-3 column, 2.1 mm×100 mm, 3 μm; and column temperature: 35 DEG C.; and

in an elution system, mobile phase A: 0.1% of formic acid in water, mobile phase B: 0.1% of formic acid in acetonitrile; flow rate: 0.2 mL/min; and gradient elution procedures;

0 to 2 min, a volume ratio of the mobile phase A to the mobile phase B is 95:5;

2 to 6 min, a volume ratio of the mobile phase A to the mobile phase B is 10:90;

6 to 8 min, a volume ratio of the mobile phase A to the mobile phase B is 10:90; and

8.5 to 14 min, a volume ratio of the mobile phase A to the mobile phase B is 95:5.

The ratio of reagents here is based on a volume ratio.

In some embodiments, in S2, a test condition for a mass spectrum includes the following parameters:

electrospray ionization in a negative mode; and a voltage of ion spray: −4000 V, and temperature: 500 DEG C.

In some embodiments, in S1, a concentration of the internal standard EtG-D₅ is 1 μg/mL, and a concentration of the internal standard EtS-D₅ is 1 μg/mL.

The technical solutions of the present disclosure achieve the following beneficial effects:

1. According to the method of the present disclosure, the drinking time is inferred mainly using a variation pattern of concentration ratios between non-oxidative metabolites of alcohol over time, such that inevitable external interference in a traditional method is avoided.

2. According to the present disclosure, a regression equation is established based on the average concentration ratio of EtG to EtS in blood and the drinking time, and thus a regression equation, y=1.646x²− 0.9599x+0.0878, R2=0.9904, in a 0-8 h window period is obtained, which indicates that a good correlation model is obtained between the average concentration ratio of EtG to EtS in blood and the time of using alcohol. The average concentration ratio of EtG to EtS is substituted into this equation to calculate, by an inverse method, a theoretical value of the drinking time. Meanwhile, inference errors are calculated using a formula “(theoretical value−measured value)/actual drinking time.” revealing that the errors are basically less than 10%.

3. According to the present disclosure, a method for calculating a length of time after drinking through pharmacokinetic studies on EtG and EtS in blood after drinking is established, and pharmacokinetic parameters of EtG and EtS in the Chinese population after appropriate oral doses are also provided. The maximum concentration, maximum concentration and elimination half life of the EtG in blood are at 4.12±1.07 h, 0.31±0.11 mg/L and 2.56±0.89 h respectively; and the maximum concentration, maximum concentration and elimination half life of the EtS are at 3.02±0.70 h, 0.17±0.04 mg/L and 2.04±0.76 h respectively.

### BRIEF DESCRIPTION OF THE DRAWINGS

FIG. 1 shows average concentration-time curves of alcohol, EtG and EtS in blood;

FIG. 2 shows liquid chromatography-mass spectrometry (LC-MS) chromatograms (500 ng/mL) of EtG, EtS, and internal standard EtG-D₅ and EtS-D₅;

FIG. 3 shows an LC-MS chromatogram of a blood-blank sample; and

FIG. 4 shows LC-MS chromatograms (500 ng/mL) of a blood-blank sample added with EtG, EtS, and internal standard EtG-D₅ and EtS-D_{5.}

### DETAILED DESCRIPTION

For clearer description of the objects, technical solutions, and advantages of the present disclosure, the present disclosure is further described in detail hereinafter with reference to the accompanying drawings and some exemplary embodiments. It should be understood that the specific embodiments described herein are merely illustrative of the present disclosure and should not be deemed as limiting the scope of the present disclosure.

Based on the embodiments of the present disclosure, all other embodiments obtained by those ordinary skilled in the art without creative efforts should fall within the scope of protection of the present disclosure.

The drinking time described in the present disclosure refers to the time from the start of drinking when the samples are taken for testing.

Unless otherwise specified, the experimental methods in the embodiments mentioned below are conventional methods, and the reagents and materials are available commercially.

### Example 1

1. Materials and methods

1.1. Chemicals and reagents

| | |
|---|---|
| Alcohol (10 mg/mL) | Accustandard, USA; |
| Tert-Butanol (AR, ≥99.0%) | Aladdin, Shanghai; |
| EtG (100 mg/mL) | Cerilliant, USA; |
| Internal standard EtG-D₅ (IS; 1 μg/mL) | Cerilliant, USA; |
| Internal standard EtS-D₅ (IS; 1 μg/mL) | Cerilliant, USA; |
| EtS-Na (98%) | TSI, Japan; |
| Methanol (HPLC grade) | Merke, USA; |
| Acetonitrile (HPLC grade) | Merke, USA; |
| Formic acid (LC/MS grade) | Bailingway, China; and |
| Ultrapure water | Milli-Q Ultrapure |
| | Water System, USA |

1.2. Participants and experimental methods

Approved by the Medical Ethics Committee of Shanxi Medical University (2018LL349), a total of 26 adults including 14 men and 12 women are recruited by the team to participate in the study. All participants have no histories of physical or mental illness, and drinking or medication, of which, a median age is 24.5 years old (in the range of 22 to 27 years old), and a mean body mass index is 20.9 kg/m² (in the range of 16.8 kg/m² to 34.6 kg/m²).

Participants signed informed consent forms prior to the start of the study. For safety, all participants were observed in a school hospital for at least 24 h upon drinking, and were medically evaluated accordingly during drinking and 3 days upon drinking.

Upon a 12-h fast, the participants drank (Fenjiu, with an alcohol content of 40%) with food within 30 minutes according to a dose standard of 0.72 g/kg (which is proportional to weights of the participants), and 5 mL of blood was drawn through indwelling catheters in median cubital veins before (0 h) drinking and 0.5 h, 1.5 h, 2 h, 3 h, 5 h, 8 h, 12 h, 24 h, 36 h, 48 h, and 120 h upon drinking respectively, as blood samples under test. All the blood samples were stored at −20 DEG C. until the end of the analysis.

1.3. Samples preparation

1.3.1. Preparation of blood samples under test containing internal standard tert-butanol

Alcohol contents in the blood samples were measured using a headspace gas chromatography internal standard method with tert-butanol as an internal standard. 1 mL of blood and 1 mL of tert-butanol (IS, 87 mg/mL) were added to a headspace vial, diluted with 3 mL of ultrapure water, mixed in a sealed condition, and then analyzed by the headspace gas chromatography.

1.3.2. Preparation of blood samples under test containing internal standards EtG-D₅ and EtS-D₅

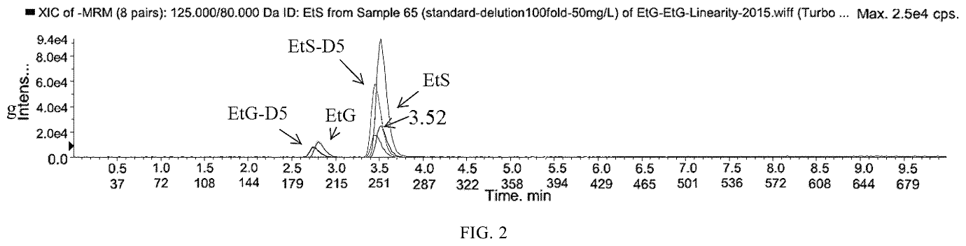

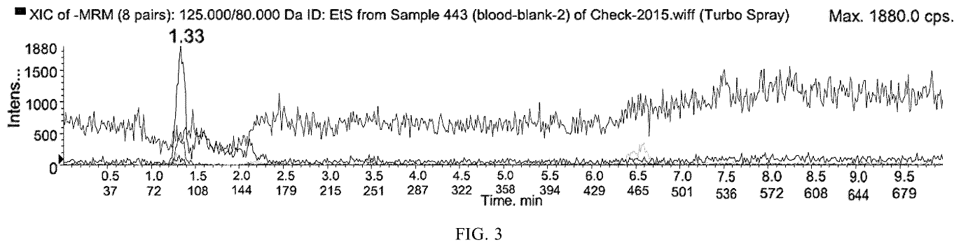

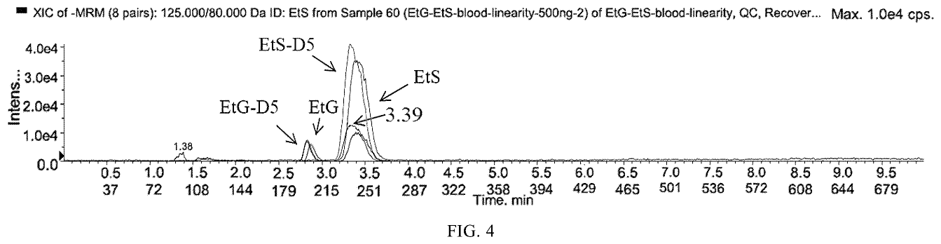

Metabolites EtG and EtS in blood samples were measured by liquid chromatography-tandem quadrupole mass spectrometry (LC-MS/MS) with EtG-D₅ and EtS-D₅ as internal standards. The internal standards EtG-D₅ (IS, 1 μg/mL) and EtS-D₅ (IS, 1 μg/mL) were taken 100 μL separately, and mixed well to obtain mixed internal standards; 100 μL of blood was taken and 100 μL of mixed internal standard was added to improve the identification and quantification of the metabolites (EtG and EtS). Then 800 μL of 80% acetonitrile in methanol was added, and the mixture is precipitated at 0 DEG C. for 10min. After that, centrifugation was performed at 13000 rpm for 5 min, supernatant was taken out, and dried, by blowing, with nitrogen at 35 DEG C., and then re-dissolved with 400 μL of 5% acetonitrile in water and centrifuged again at 13000 rpm for 5 min. 3 μL of the supernatant was taken and injected into the LC-MS/MS for analysis, as shown in FIGS. 2 to 4.

1.4 Mass spectrometry analysis

Chromatograph separation was performed via an LC-20 A system. Conditions of the chromatograph were as follows:

chromatographic column: Inertsil ODS-3 column (2.1 mm×100 mm, 3 μm; Shimadzu, Japan) , and column temperature: 35 DEG C.

Mobile phases: mobile phase A (0.1% of formic acid in ultrapure water) and mobile phase B (0.1% of formic acid in acetonitrile); gradient elution (see Table 1); flow rate: 0.2 mL/min; total time of elution: 14.0 min; and injection volume: 5 μL.

TABLE 1
Gradient elution conditions

| Time/min | A/% | B/% |
|---|---|---|
| 0-2.0 | 95 | 5 |
| 2.0-6.0 | 10 | 90 |
| 6.0-8.0 | 10 | 90 |
| 8.0-8.5 | 95 | 5 |
| 8.5-14.0 | 95 | 5 |

Targeted substances were tested by a tandom mass spectrometer (TRAP4,000, Sciex, AB). The specific conditions were as follows:

Ion source: electron spray ionization (ESI); voltage of ion spray: −4000 V, and temperature: 500 DEG C.; curtain gas, nebulizer (Gas 1), and heating auxiliary gas (Gas 2): 40 psi, 50 psi, and 35 psi respectively.

Scanning mode: anions+multiple reaction monitoring (MRM).

Other specific MRM parameters for each analyte are shown in Table 2.

TABLE 2
Characteristic ion pairs and mass spectrometric
data for each analyte

| Targeted | Qualitative ions | Quantitative | Declustering | Collision |
|---|---|---|---|---|
| substances | (m/z) | ions (m/z) | potential (V) | energy (V) |
| EtS | 125.0/80.0 | 125.0/97.0 | 46 | 45 |
| | 125.0/97.0a | | | 21 |
| EtG | 221.1/75.0a | 221.1/75.0 | 63 | 22 |
| | 221.1/85.0 | | | 23 |
| EtG-D₅ | 226.1/75.0a | 226.1/75.0 | 63 | 23 |
| | 226.1/85.0 | | | 26 |
| EtS-D₅ | 130.0/80.0a | 130.0/80.0 | 46 | 46 |
| | 130.0/97.9 | | | 25 |

Note:
a represents a quantitative ion pair.

1.5. Estimation of the last drinking time

The last drinking time was estimated based on a relationship between the average concentration ratio C_{EtG}/C_{EtS} of EtG to EtS and the sampling time, and an error between observed time and actual time was calculated according to the following formula:

Error=(observed value−actual value)/actual value)*100%.

In the formula, the observed value represents theoretical observed time, that is, the estimated time of the last drinking; and the actual value represents the actual time, that is, actual sampling time since the last drinking.

1.6. Statistics

The pharmacokinetic parameters were calculated by a non-compartmental model using a DAS 3.0 software. All data was summarized using descriptive statistics. Some key data including arithmetic mean values and standard deviations of targeted substance concentrations, detection time points, pharmacokinetic parameters and other results were provided. All statistical analyses were performed using version 13.0 of an IBM SPSS® software (SPSS Inc., Chicago, IL, USA).

2. Results

2.1. Verification of the method

Limit of detection (LOD) and limit of quantitation (LOQ) for the EtG and EtS in blood samples were 0.02 μg/mL and 0.05 μg/mL respectively. Residue obtained from previous treatment was resolved with 100 μL of 5% acetonitrile aqueous solution to quantify concentrations below LOQ.

TABLE 3
Linear ranges and LOD of EtG and EtS in blood

| Targeted | Linear | | | | LOD | LOQ |
|---|---|---|---|---|---|---|
| sub- | ranges | | | | (μg/ | (μg/ |
| stances | (μg/mL) | Linear equation | R² | Weight | mL) | mL) |
| EtG | 0.05 − 5.0 | y = 1.7411x + 0.0036 | 0.9999 | Non | 0.02 | 0.05 |
| EtS | 0.05 − 5.0 | Y = 1.803x − 0.0279 | 0.9997 | Non | 0.02 | 0.05 |

TABLE 4
Precision, recovery and matrix effects of EtG and EtS in blood

| Targeted | Con- | Within-batch | Between-batch | Re- | Matrix |
|---|---|---|---|---|---|
| sub- | centration | precision | precision | covery | effect |
| stances | (μg/mL) | (%) | (%) | (%) | (%) |
| EtG | 0.05 | 5.2 | 4.8 | 70.1 | 15.4 |
| | 0.5 | 4.8 | 2.4 | 66.9 | 11.5 |
| | 5.0 | 4.7 | 2.0 | 66.1 | 11.9 |
| EtS | 0.05 | 4.0 | 5.7 | 85.1 | 3.9 |
| | 0.5 | 4.7 | 2.0 | 82.0 | 1.0 |
| | 5.0 | 2.5 | 6.1 | 88.6 | 0.9 |

As shown in Tables 3 to 4, all analytes including alcohol, EtG, EtS, EtG-D₅ and EtS-D₅ are well separated and no endogenous peaks are eluted by the analytes, such that the method is verified fully. 2.2. Estimation of the last drinking time

TABLE 5
Average concentrations (x ± S (min − max), n = 26) of alcohol
and metabolites thereof in human blood

| Time | BAC (mg/mL) | EtG (μg/mL) | EtS (μg/mL) |
|---|---|---|---|
| 0 | — | — | — |
| 0.5 h | 0.34 ± 0.10 | 0.05 ± 0.02 | 0.06 ± 0.02 |
| | (0.19 − 0.61) | (0.02 − 0.08) | (0.03 − 0.09) |
| 1.5 h | 0.41 ± 0.11 | 0.14 ± 0.04 | 0.11 ± 0.02 |
| | (0.22 − 0.66) | (0.06 − 0.24) | (0.06 − 0.16) |
| 2 h | 0.41 ± 0.12 | 0.20 ± 0.06 | 0.14 ± 0.03 |
| | (0.14 − 0.60) | (0.10 − 0.34) | (0.07 − 0.20) |
| 3 h | 0.36 ± 0.14 | 0.27 ± 0.09 | 0.16 ± 0.04 |
| | (0.07 − 0.63) | (0.13 − 0.47) | (0.07 − 0.25) |
| 5 h | 0.17 ± 0.10 | 0.29 ± 0.12 | 0.14 ± 0.05 |
| | (0.00 − 0.37) | (0.09 − 0.53) | (0.03 − 0.24) |
| 8 h | 0.03 ± 0.04 | 0.14 ± 0.08 | 0.06 ± 0.03 |
| | (0.00 − 0.14) | (0.04 − 0.30) | (0.01 − 0.11) |
| 12 h | — | 0.04 ± 0.03 | 0.02 ± 0.01 |
| | | (0.01 − 0.12) | (0.00 − 0.04) |

Note:
“—” represents “not detected”; BAC represents blood alcohol concentration; and all values are accurate to two decimal places. The alcohol, EtG and EtS are not detected at 24 h, 36 h, 48 h, and 120 h upon drinking. The intervals for collecting blood samples are selected from 0 to 120 h, which is based on the fact that a detection window period of non-oxidative metabolites of alcohol are longer than that of elementary bodies of alcohol reported in the literature. However, in the detection process of the embodiments of the present disclosure, it is found that the individual targeted substance is undetectable after 24 h.

According to the average concentration of the EtG and EtS in blood samples as shown in Table 5, the average concentration ratio C_{EtG}/C_{EtS} of EtG to EtS is calculated, and the relationship between the ratio after a single oral dose and the last time of use is analyzed, showing that a quadratic regression equation y=1.646x²−0.9599x+0.0878, R²=0.9904, is obtained by using the average concentration ratio C_{EtG}/C_{EtS} as an abscissa and the sampling time as an ordinate. In the formula, x represents the average concentration ratio C_{EtG}/C_{EtS}, and y represents the sampling time.

TABLE 6
Errors between the time deduced from a quadratic
function and the actual last drinking time

| C_{EtG}/ | Observed value | Actual | Error |
|---|---|---|---|
| C_{Ets} | (h) (CI) | value (h) | (%) |
| — | 0.00 | 0.00 | 0.00 |
| 0.79 | 0.35 (0.27 − 0.63) | 0.50 | 29.05 |
| 1.22 | 1.36 (1.16 − 2.04) | 1.50 | 9.06 |
| 1.42 | 2.03 (1.64 − 3.02) | 2.00 | 1.41 |
| 1.65 | 2.99 (2.68 − 4.86) | 3.00 | 0.18 |
| 2.13 | 5.53 (5.04 − 8.43) | 5.00 | 10.54 |
| 2.45 | 7.64 (6.57 − 11.52) | 8.00 | 4.46 |

CI represents a confidence interval (95%).

As shown in Table 6, the concentration ratio of EtG to EtS is substituted into the regression equation (y=1.646x²−0.9599×+0.0878, in which x represents ratio, y represents time and R²=0.9904) to calculate the observed value of the drinking time. The error between the observed value and the actual value within 8 h is obtained using the error calculation formula (error=(observed value−actual value)/actual value)* 100%), and the errors are basically less than 10%.

2.3. Pharmacokinetic analysis

The average concentrations of the alcohol and metabolites thereof in human blood at each time point are shown in Table 5, and LOD data for the alcohol and metabolites thereof are shown in Table 7.

TABLE 7
LOD data (x ± S (min − max), n = 26) for alcohol and
metabolites thereof in human blood

| Targeted substances | Alcohol | EtG | EtS |
|---|---|---|---|
| LOD (h) | 5.81 ± 1.74 | 22.15 ± 4.42 | 16.92 ± 6.23 |
| | (3.00 − 8.00) | (12.00 − 24.00) | (8.00 − 24.00) |

The result shows that after drinking 0.72 g alcohol/kg, the average blood alcohol concentration of the participants reaches 0.41±0.11 mg/mL 1.5 h latter, and then gradually decreases, with a detection window time (maximum observed value) of 3 to 8 h.

The metabolites EtG (0.29±0.12 μg/mL) and EtS (0.16±0.04 μg/mL) reach peak values at 5 h and 3 h respectively.

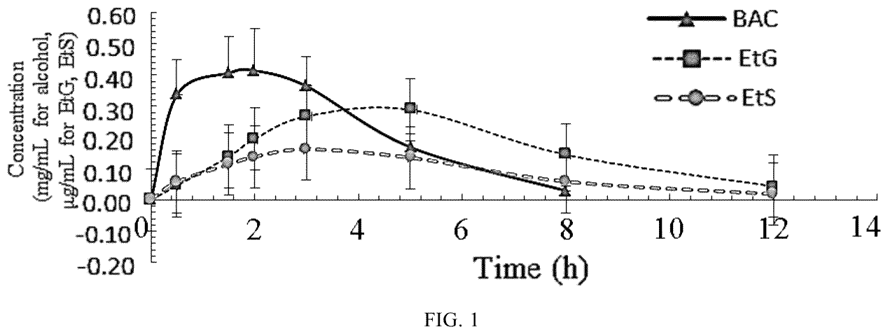

In addition, as shown in FIG. 1, in the study process, it is found that the concentration of EtG is consistently higher than that of EtS.

Based on the non-compartmental model, after the participants drink 0.72 g alcohol/kg, the pharmacokinetic parameters for the alcohol in blood as well as the metabolites EtG and EtS are calculated, to obtain a pharmacokinetic model, and the result is shown in Table 8.

TABLE 8
Pharmacokinetic parameters (x ± S, min − max, n = 26) for
alcohol and metabolites thereof in human blood

| Parameters | Samples containing alcohol | EtG | EtS |
|---|---|---|---|
| AUC (0-t) | 1,715.23 ± 626.72 | 1.99 ± 0.78 | 1.04 ± 0.34 |
| (mg/L*) | (505.00 − 2,914.00) | (0.76 − 3.55) | (0.41 − 1.75) |
| t1/2z (h) | 0.241 ± 1.09 | 2.56 ± 0.89 | 2.04 ± 0.76 |
| | (0.30 − 4.23) | (1.03 − 4.94) | (1.11 − 3.32) |
| Tₘₐₓ (h) | 2.02 ± 0.54 | 4.12 ± 1.07 | 3.02 ± 0.70 |
| | (1.50 − 3.00) | .00 (2 − 5.00) | (1.50 − 5.00) |
| Cₘₐₓ (mg/L) | 441.65 ± 113.86 | 0.31 ± 0.11 | 0.17 ± 0.04 |
| | (238.20 − 656.00) | (0.13 − 0.53) | (0.08 − 0.28) |
| Vz/F (L/kg) | 0.69 ± 0.49 | — | — |
| | (0.11 − 1.76) | | |
| Clz/F (L/h) | 0.49 ± 0.33 | — | — |
| | (0.17 − 0.43) | | |

Note:
AUC (0-t) represents an area under the curve;
t1/2z represents a half-life period;
Tₘₐₓ represents time to peak;
Cₘₐₓ represents a peak concentration;
Vz/F represents apparent volume of distribution; and
Clz/F represents a clearance rate.

The result shows that the peak concentration (441.65±113.86 mg/L (0.44±0.11 mg/mL)) of the alcohol is reached at 2.02±0.54 h. The peak concentrations (0.31±0.11 mg/L and 0.17±0.04 mg/L) of the metabolites are reached at 4.12±1.07 h and 3.02±0.70 h. T1/2z of alcohol, EtG and EtS are at 1±1.09 h, 2.56±0.89 h and 2.04±0.76 h. Clz/F of alcohol is at 0.49±0.33 L/h. However, due to in-vivo doses of metabolites (EtG and EtS) of alcohol are unknown, the Vz/F and CLz/F for both cannot be accurately calculated.

3. Discussion

According to the present disclosure, the drinking time is inferred, mainly based on the pharmacokinetic study, using a variation pattern of the average concentration ratio between non-oxidative metabolites of alcohol over time. Specifically, a regression equation is established based on the average concentration ratio of EtG to EtS in blood and the drinking time, and thus a regression equation y=1.646x²−0.9599x+0.0878, R2=0.9904 in a 0-8 h window period is obtained, which indicates that the average concentration ratio of EtG to EtS in blood has a good correlation with the time of using alcohol. The average concentration ratio of EtG to EtS is substituted into this equation to calculate a theoretical value of the drinking time using an inverse method. Meanwhile, inference errors are calculated using the formula “(theoretical value−measured value)/actual drinking time,” revealing that the errors are basically less than 10%.

The LOD and LOQ of the non-oxidative metabolites (EtG and EtS) of alcohol in blood samples are 0.02 μg/mL and 0.05 μg/mL respectively, indicating that the method of the present disclosure can effectively quantify the EtG and EtS with lower concentrations in blood. The blood alcohol concentration (BAC) of 0.72 g/kg alcohol dose in the embodiments of the present disclosure is in the range of 0.22 to 0.66 mg/mL, which is similar to the existing BAC standard for determining drunk driving (>0.2 mg/mL), indicating that the method of the embodiments of the present disclosure is applicable to monitoring of most drunk driving cases in China.

According to the present disclosure, based on the non-compartmental model, the pharmacokinetic parameters of the alcohol, EtG and EtS in blood are calculated, which indicates that the peak concentration Cₘₐₓ (441.65±113.86 mg/L (0.44±0.11 mg/mL)) of the alcohol is reached at 2.02±0.54 h. Compared with previous studies, an absorption phase of the alcohol obtained by the embodiments of the present disclosure is longer, that is, the absorption is slower. Furthermore, it is found that alcohol could be detected in the participants' blood within 3 to 8 h and the average elimination half life of the alcohol is at 1.24±1.09 h (0.30 to 4.23 h).

According to the present disclosure, based on the non-compartmental model, the pharmacokinetic parameters of the alcohol, EtG and EtS in blood are calculated, which indicates that the metabolites EtG and EtS have longer detection window periods, and confirms that metabolism velocity of EtG is slower than that of alcohol, and the elimination half life of EtG in blood is at 2.56±0.89 h. EtS is another non-oxidative metabolite of alcohol metabolism with a concentration-time curve similar to that of EtG. In the studies, at a dose of 0.72 g/kg, the peak concentration Cₘₐₓ of EtS is 0.17 μg/mL (in the range of 0.08 μg/mL to 0.28 μg/mL), and the peak time Tₘₐₓ is at 3.02 h. In addition, it is also found that the detection window period and peak concentration Cₘₐₓ of EtS are significantly lower than those of EtG. However, EtS is more stable and insensitive to bacteria. Accordingly, the EtS can provide supplementary data for identifying alcohol intake.

In conclusion, according to the studies of the present disclosure, an idea and method for inferring the drinking time using the average concentration ratio EtG/EtS, which, after further verification, are expected to provide a useful analytical monitoring tool for drunk-driving identification and related inference of the drinking time in China. Moreover, pharmacokinetics of EtG and EtS in blood of Chinese population are further studied, and the pharmacokinetic parameters for both are obtained. The sensitive LC-MS/MS method developed and verified in the embodiments of the present disclosure can be applied to drunk-driving and other forensic cases involving alcohol. The long detection window periods of EtG and EtS support the EtG and EtS as useful markers for detecting alcohol consumption.

The above described are merely the preferred embodiments of the present disclosure, not used for limiting the present disclosure. Any modifications, equivalent replacements, improvements and the like made within the spirit and principles of the present disclosure should be included in the scope of protection of the present disclosure.

1. A method for calculating drinking time, comprising:
drawing a plurality of blood samples within 0 to 120 h upon start of drinking, testing concentrations of EtG and EtS in the blood samples, and obtaining an average concentration ratio C_{EtG}/C_{EtS} of EtG to EtS;
obtaining a quadratic regression equation: y=1.646x²−0.9599x+0.0878, R²=0.9904 by fitting using the average concentration ratio C_{EtG}/C_{EtS} as an abscissa and sampling time as an ordinate,
wherein x represents the average concentration ratio C_{EtG}/C_{EtS}, and y represents the sampling time; and
measuring C_{EtG}/C_{EtS} of blood samples under test, obtaining a relationship between the drinking time and the C_{EtG}/C_{EtS} based on the quadratic regression equation, and calculating the drinking time.

2. The method for calculating drinking time according to claim 1, wherein a blood alcohol concentration upon drinking is in the range of 0.22 to 0.66 mg/m.

3. The method for calculating drinking time according to claim 2, wherein an alcohol intake amount is 0.72 g/kg.

4. The method for calculating drinking time according to claim 1, wherein sampling intervals of the blood samples used for obtaining the quadratic regression equation are at 0 h, 0.5 h, 2 h, 3 h, 5 h, 8 h, 12 h, 24 h, 36 h, 48 h, and 120 h respectively.

5. The method for calculating drinking time according to claim 1, wherein the concentrations of EtG and EtS in the blood samples are tested by:
S1. pre-treating the blood samples
transferring the blood samples into centrifuge tubes added with internal standards EtG-D₅ and EtS-D₅, adding 80% of acetonitrile in methanol, precipitating and centrifuging at 0 DEG C., transferring supernatant, drying, re-dissolving with 5% of acetonitrile in water, centrifuging again, and taking the supernatant to obtain the blood samples under test; and
S2. measuring concentrations of EtG and EtS by liquid chromatography-tandem mass spectrometry for the blood samples under test in S1.

6. The method for calculating drinking time according to claim 5, wherein in S2, a separation condition for liquid chromatography comprises the following parameters:
chromatographic column: an Inertsil ODS-3 column, 2.1 mm×100 mm, 3 μm; and column temperature: 35 DEG C.; and
in an elution system, mobile phase A: 0.1% of formic acid in water, mobile phase B: 0.1% of formic acid in acetonitrile; flow rate: 0.2 mL/min; and gradient elution procedures:
0 to 2 min, a volume ratio of the mobile phase A to the mobile phase B is 95:5;
2 to 6 min, a volume ratio of the mobile phase A to the mobile phase B is 10:90;
6 to 8 min, a volume ratio of the mobile phase A to the mobile phase B is 10:90; and
8.5 to 14 min, a volume ratio of the mobile phase A to the mobile phase B is 95:5.

7. The method for calculating drinking time according to claim 5, wherein in S2, a test condition for a mass spectrum comprises the following parameters:
electrospray ionization in a negative mode; and
voltage of ion spray: −4000 V, and temperature: 500 DEG C.

8. The method for calculating drinking time according to claim 5, wherein in S 1, a concentration of the internal standard EtG-D₅ is 1 μg/mL, and a concentration of the internal standard EtS-D₅ is 1 μg/mL.
